(19) United States
(12) Patent Application Publication
(10) Pub. No.: US 2002/0142295 A1
(43) Pub. Date: Oct. 3, 2002

(54) SEQUENCE-BASED MUTATION ANALYSIS OF NEOPLASTIC TISSUE FOR DIAGNOSIS OR PROGNOSIS OF THE NEOPLASIA

(76) Inventors: MARGARET BYWATER, PHILADELPHIA, PA (US); PER LINDSTROM, UPPSALA (SE); MATS INGANAS, UPPSALA (SE)

Correspondence Address: BIRCH STEWART KOLASCH & BIRCH, PO BOX 747, FALLS CHURCH, VA, 22040-0747, US

(21) Appl. No.: 08/776,044

(22) PCT Filed: Jun. 29, 1995
(86) PCT No.: PCT/SE95/00804

(30) Foreign Application Priority Data
Jul. 15, 1994 (SE) 9402487-4
Nov. 16, 1994 (SE) 9403953-4

(51) Int. Cl. C12Q 1/68

(52) U.S. Cl. 435/6

(57) ABSTRACT

A method for sequence-based diagnosis of a human neoplastic tissue, blood or other body fluid sample, comprises analysing from genomic DNA or cDNA derived from said neoplasia the DNA sequence of a gene encoding a cancer-related protein for the presence of mutations therein, determining from the presence, nature and location of any such mutation or mutations the influence thereof on the biological function of the corresponding protein and thereby on the properties of the neoplasia, and on the basis thereof prognosticating the development of the neoplasia.

### TECHNICAL AREA OF THE INVENTION

The present invention relates to the area of cancer diagnostics. More particularly, the invention relates to the detection of alteration in cancer-related genes derived from a neoplasia sample and the use thereof for prognostic purposes.

### BACKGROUND OF THE INVENTION

Breast cancer is the most common cancer in women. Although it is recognized that breast cancer tends to run in families, unpredictable acquired somatic mutations are responsible for the large majority of cases. There is today an underlying controversy in the prediction of outcome when a woman is diagnosed as having breast cancer. Thus, when a lesion is discovered in a woman's breast, the diagnosis, cancer or not, is carried out on the basis of morphological change of the tumour and surrounding tissue. However, the prognosis or outcome influences the clinician's choice of treatment considerably. Prognostic factors can be divided into two categories, i.e. biological and chronological factors.

The determination of biological factors include cytological examination of a needle biopsy of the tumour. Immunohistochemical staining is used to investigate the presence and quantity of hormone receptors, and DNA labelling methods quantify the amount of DNA in the cells and DNA synthesis. Chronological factors include tumour size and axillary nodal status, the latter being the traditional prognostic factor in the management of breast cancer.

In case cancer is diagnosed, the 20-30 lymph nodes are removed surgically, and the number of nodes containing cancer cells are counted. If more than a finite number of nodes (e.g. five) is identified, the patient is exposed to radical treatment, surgically as well as radiation/polychemotherapy or both. While the biological factors are being increasingly used to make treatment decisions of the disease, lymph node status remains the standard against which the predictive power of biological prognostic factors are evaluated.

It is believed that patients with breast cancer that have axillary lymph node involvement have a relatively poor prognosis, partly due to other biological factors effecting aggressiveness and/or metastatic potential of the tumour, irrespective of the chronological stage at which the tumour is investigated. Recent findings suggest, however, that the presence and extent of lymph node metastasis has little to do with tumour aggressiveness or metastatic potential, but is entirely a reflection of the relatively advanced chronological age of the tumor.

In the absence of more relevant prognostic factors of the outcome of the disease, most clinicians still rely on the clinical or chronological factors together with the morphological grade of the tumour in the microscope, factors which by the way have been used over 40 years.

There is therefore a need for more accurate indication of the biological status in the form of aggressiveness and metastatic potential available at early diagnosis which would enable the clinician to take steps to treat the patient earlier and more accurately. Thus, a small tumour with metastatic potential could be treated with radical methods already from the initial diagnosis instead of waiting for relapse. Moreover, patients who do not have a tumour with metastatic potential where the risk for relapse is very small or non-existent, could be treated with milder methods.

During the last few years, research efforts have been directed to the finding of correlations between genetic mutation and cancer development and progression. An interesting type of genes in this context are the tumour suppressor genes, which are defined as genes for which loss-of-function mutations are oncogenic. Wild-type alleles of such genes may function to prevent or suppress oncogene. An example of such a gene is the p53 gene on chromosome 17p which encodes the tumour suppressor protein p53. Mutations in the p53 gene can be found in about half of all cases of human cancer. Cancer forms which have been found to have a strong correlation with mutations in the p53 gene are, for example, breast cancer and colon cancer. A method of diagnosing human neoplasia or cancer, such as breast, colorectal or lung cancer, by detecting loss of wild-type p53 genes in a sample suspected of being neoplastic is disclosed in EP-A-390 323.

The kinds of mutations that make the tumour suppressor genes defective vary between different tumour suppressor genes. Thus, whereas the tumour suppressor genes which are defective in e.g. retinoblastoma are commonly inactivated by nonsense mutations that cause truncation and instability of the protein, approximately 70% of the mutations in p53 are missense mutations that change the identity of an amino acid. Such amino acid changes can alter the conformation and thereby the stability of the p53 protein and can indirectly alter the sequence-specific DNA binding and transcription factor activity of the p53.

Recent results show that p53 plays an important role in the control of DNA repair mechanisms, preventing DNA replication prior to cell division until repair is completed. It has also been found that there are hot-spots in the gene that are more prone to mutation, but the mutations are in general acquired randomly and spontaneously within the hot-spot regions.

As far as breast cancer is concerned, a correlation has been observed between survival and p53 mutation. Thus, Thorlacius et al., Cancer Res. 53 (1993) 1637-1641 report that women with a p53 mutation in the breast tumour run a more than threefold higher risk of dying than those without a p53 mutation.

Apart from the above correlation with survival, however, analysis of p53 mutations in breast tumours as well as in other tumours has failed to establish any correlation with clinical parameters and prognosis in other respects.

### SUMMARY OF THE INVENTION

It is an object of the present invention to provide a more reliable method for diagnosing human neoplastic tissue, blood or other body fluid, based on the detection of possible mutations in a cancer-related gene, such as the p53 gene, with regard to biological status in the form of aggressiveness and metastatic potential to enable an accurate prediction of the disease and thereby permit the clinician to classify the patients with respect to the determined biological status of the tumour and provide an adequate treatment depending on the classification, especially at early diagnosis.

In accordance with the invention, it has now been found that by determining from a human neoplasia sample from an afflicted patient the location and nature of a mutation in a cancer-related gene, the severity of the detected changes for the outcome of the patient may be evaluated and the treatment may be adapted thereto. Of particular relevance is the detection of the location and nature of mutations in those parts of the gene which encode a biologically functional domain or domains of the protein.

The invention therefore provides a method of diagnosing a human neoplasia in a tissue, blood or other body fluid sample (e.g. urine, sputum), which comprises analysing from genomic DNA or cDNA derived from said neoplasia the DNA sequence of a gene encoding a cancer-related protein for the presence of mutations therein, determining from the presence, nature and location of any such mutation or mutations the influence thereof on the biological function of the corresponding protein and thereby on the properties of the neoplasia, and on the basis thereof prognosticating the development of the neoplasia and provide a guidance for adequate treatment of the patient.

The expression “cancer-related gene”, as used herein means any gene for which a mutation may be correlated with the development of neoplasia or cancer. Such genes generally encode proteins taking part in the DNA replication cycle, such as suppressor proteins, oncogens including growth inducing proteins, and regulatory proteins. Exemplary of such genes are, besides the p53 gene already mentioned above, those encoding the proteins WAFI, erb B-2 (HerII/Neu), p16 (MTS I), MTS II, MLH 1 & 2 and Ras.

The mutations to be detected include point mutations, deletions and insertions as well as polymorphisms.

The present invention also provides specific primers for amplification and sequencing, respectively, of p53 genomic and cDNA.

### BRIEF DESCRIPTION OF THE DRAWINGS

FIG. 1 is a schematic representation of the p53 protein, wherein the locations of the evolutionary conserved regions as well as the transactivation domain (A), the DNA binding domain (B) and the oligomerization domain (C) are indicated

FIG. 2 is a schematic representation of p53 cDNA with aligned coding region as well as four amplified and sequenced overlapping fragments thereof used in Example 1 below. On the fragments 1 to 4, primers are indicated by “←”. “B” indicates a biotinylated primer and “S ” indicates a sequencing primer.

FIG. 3 is a similar representation to that in FIG. 2 but with different fragments and primers, also used in Example 1 below.

FIG. 4 is a graph (“survival plot”) showing relapse-free survival after surgery of node negative breast cancer patients without p53 mutation who (i) received and (ii) did not receive adjuvant therapy.

FIG. 5 is a similar graph to that in FIG. 4 for node negative breast cancer patients with p53 mutation.

FIG. 6 is a similar graph to that in FIG. 4 showing relapse-free survival after surgery of node positive breast cancer patients (i) with p53 mutation and (ii) without p53 mutation.

FIG. 7 is a graph (“survival plot”) showing relapse-free survival after surgery of node negative breast cancer patients without p53 mutation who (i) received and (ii) did not receive loco-regional radiotherapy.

FIG. 8 is a similar graph to that in FIG. 7 for node negative breast cancer patients with p53 mutation.

FIG. 9 is a graph (“survival plot”) showing relapse-free survival of breast cancer patients with a p53 mutation in conserved region II versus breast cancer patients with a mutation outside conserved regions.

FIG. 10 is a similar graph to that in FIG. 9 for breast cancer patients with a p53 mutation in conserved region III versus breast cancer patients with mutations outside conserved regions.

FIG. 11 is a similar graph to that in FIG. 9 for breast cancer patients with a p53 mutation in conserved region IV versus breast cancer patients with mutations outside conserved regions.

FIG. 12 is a similar graph to that in FIG. 9 for breast cancer patients with a p53 mutation in conserved region V versus breast cancer patients with mutations outside conserved regions.

FIG. 13 is a bar chart representation showing the location of mutations in the coding sequence of p53 for a number of breast cancer patients. The height of the bars indicate the number of patients with each mutation.

FIG. 14 is a similar bar chart to that in FIG. 13 for node negative patients. Also relapse (o) and death in breast cancer (o) is indicated in this chart, when relevant.

FIG. 15 is a similar bar chart to that in FIG. 14 for node positive patients.

### DETAILED DESCRIPTION OF THE INVENTION

The p53 protein structure as well as various mutations detected therein have been described inter alia by Harris, C., Science 262 (1993) 1980-1981. As shown therein, p53 has has a transactivation domain, an oligomerization domain, and five evolutionary conserved regions. Yunje, C. et al., Science 265 (1994) 346-354 describes the crystal structure of a complex containing the core domain of human p53 and a DNA binding site. The complete DNA sequence of the normal or wild type p53 gene may be found in, for example, Zakut-Houri, R., et al., EMBO J. 4 (1985) 1251-1255, GenBank, entry HUMP53C (cDNA sequence), as well as in Mol. Biol. Cell. 6 (1986) 1379-1385 and Mol. Cell. Biol. 7 (1987) 961-963, EMBL database, entry HSP53G (genomic DNA sequence).

In accordance with the invention, it has now been found that, especially with respect to breast cancer, there is a relationship between (i) the position of the mutation in p53 cDNA, (ii) the evolutionary conserved and functional region in the protein, and (iii) the amino acid transition.

It has also been found that, in general, a mutation or mutations in the p53 gene mediate a poor prognosis for the breast cancer patient, irrespective of other biological factors, like hormone receptor status or lymph node involvement at the initial presentation.

For example, a mutation(s) in the p53 gene located in the evolutionary conserved regions in or close to the DNA binding functional domain of the p53 protein mediate a lower affinity binding to the specific motif or a non-specific binding to other regulatory motifs, thus effecting the expression of other genes in the DNA pathway.

Likewise, a p53 mutation(s) located in the conserved regions close to the transactivation site in the p53 protein have in several cases given rise to a transcriptional stop signal which results in a truncated protein which lacks the transactivation site. This will “knock out” the protein in its role a block in cell division while DNA proof-reading and repair takes place. The tumour cells will thereby be anarchistic, resulting in a fast growing aggressive tumour.

Thus, by analysing the distribution of the mutations by DNA sequencing at least throughout the part or parts of the p53 gene which encode biologically functional domains, it is possible to distinguish between (i) mutations detrimental to the patient, e.g., such affecting the DNA binding or transactivation, and (ii) mutations less harmful for the patient, i.e. amino acid changes not greatly effecting structure or function.

More particularly, it has now been found that, in general, breast cancer patients may be classified into subgroups with regard to the position and nature of the mutation(s) and the consequential requirements on the treatment or therapy of the patient.

Thus, one large subgroup (about half of the studied patients) consists of node negative patients without p53 mutations. To these patients, today's adjuvant radiation or polychemotherapy/hormone therapy after surgical removal of the tumour does not seem to have any effect. In other words, patients who receive adjuvant therapy do not exhibit any better prognosis than those who do not receive adjuvant therapy.

Another subgroup consists of node negative patients with p53 mutations. These patients have been found to have a poor prognosis but perform very well if given appropriate adjuvant therapy. In a special study it was found that these patients had a significantly improved survival when treated with loco-regional radiotherapy. The possibility offered by the present invention to identify this subgroup of breast cancer patients is therefore of great value.

Still another subgroup consists of node positive breast cancer patients with p53 mutations. These patients have been found to have a very poor prognosis even when given today's adjuvant therapy. A more efficient therapy is therefore required for this subgroup, such as, for example, autologous bone marrow transplant.

Yet another subgroup, finally, consists of node positive breast cancer patients without p53 mutations. These patients have been found to have a better prognosis than node positive patients with p53 mutations, and today's adjuvant therapy does not seem to have any effect on the survival rate of these patients.

It is to be noted that the above described classification of breast cancer patients and the implications thereof on the therapy to be given can not be made with other analytical systems, such as immunohistochemical (IHC) staining procedures which are based on immunochemical detection of p53 expression as indicative of p53 mutation.

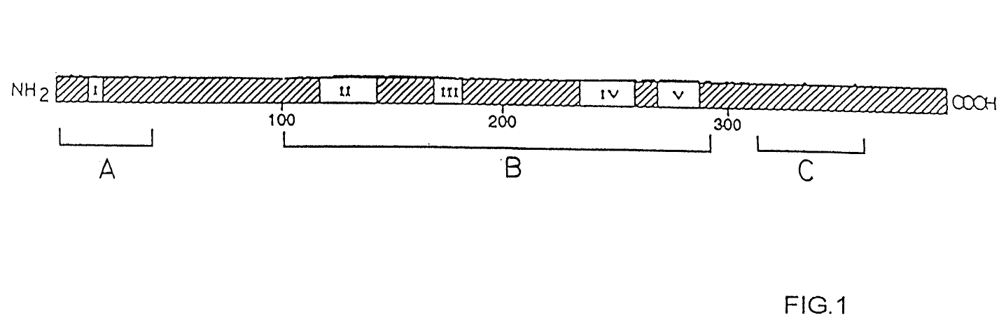

As mentioned above, the position of a mutation in the p53 gene is also decisive for the prognosis of the patient. Thus, whereas mutations in the conserved regions II and V (see FIG. 1) generally are serious, mutations in conserved regions III and IV seem to be of a less serious nature.

By determining the DNA sequence of the p53 gene in a malignant sample and classifying the mutations with respect to tumour aggressiveness and metastatic potential in accordance with the above, the clinician will thus be provided with a reliable prognostic factor correlating to the incidence of relapse. The treatment of a breast cancer patient, in the form of minor or radical surgery, with or without radiation and/or adjuvant polychemotherapy, can then be designed accordingly. For example, as mentioned above, patients lacking other alarming factors but with a p53 mutation in a critical region, who today would be subjected to milder treatment forms, could be subjected to radical treatment already from the first diagnosis. Likewise, women with e.g. lymph node involvement but with a non-critical p53 mutation, who today would receive radical treatment, could have a milder treatment. This would, of course, have an effect on both treatment costs and unnecessary suffering.

What has been said above about mutations in p53 and breast cancer is, of course, also applicable to neoplastic changes in other organs, such as lung, prostate, gastric, bladder and colorectal cancer as well as leukemia and malignant melanoma. Similarly, the inventive concept is applicable to other cancer-related genes than the p53 gene as described above.

Methods for sample preparation and DNA sequencing and data interpretation are known per se in the art and will therefore not be particularly described herein. An innovative method for the handling of multiple clinical samples for analysing a gene for mutations, which method, especially with respect to the p53 gene, is a separate aspect of the present invention, comprises the following steps:

(i) sample preparation,

(ii) amplification of genomic DNA or cDNA,

(iii) processing of the amplified product(s), preferably using a solid phase technique,

(iv) detection on an automated sequencer, and optionally

(v) use of computer software to track and control the sample and process steps and/or to aid in and/or interpret the sequence data obtained.

In the sample preparation step, either genomic DNA is prepared or cDNA is prepared from mRNA.

Amplification of the DNA is preferably performed by PCR, although other amplification techniques are, of course, also conceivable. In the case of PCR, one of the primers is preferably provided with a “separation handle”, e.g. a biotinyl group.

In the solid phase processing of the amplified DNA, the DNA fragments are captured on a solid support, such as by binding of a biotinylated DNA fragment to a solid support with immobilized avidin or streptavidin. After melting off the non-biotinylated DNA strand, the sequencing primers are annealed to the immobilized DNA fragments and sequencing reactions with the four dNTP's and respective terminators, such as ddNTP's, are performed with the immobilized-DNA fragments as templates, as is per se known in the art.

The primer extension products are then eletrophoretically separated and detected on an automated nucleic acid sequencer.

Preferably, especially with respect to the p53 gene, several overlapping fragments are amplified and sequenced.

The solid support may be in bead form, such as magnetic beads. A preferred solid phase processing system is, however, disclosed in our WO 94/00597 and WO 94/11529 (the entire disclosures of which are incorporated by reference herein) and comprises a multi-pronged device, usually a comb-like element, the pin tips or teeth of which constitute the immobilization surfaces.

Computer software may be used on two levels, (i) for tracking the different samples throughout the processing and analysis and controlling the different process steps, and (ii) for at least aiding in the interpretation of the sequence data obtained.

Hereinafter, the invention will be illustrated by the following non-limiting examples.

### EXAMPLE 1

Tumour samples from a first group of 107 and a second group of 292 breast cancer patients with identified node status (node negative or node positive) were prepared and sequenced as follows.

Preparation of MRNA from Patient Sample

300 μl of RNAzole™ (phenol and GTC, Cinna/Biotecx Lab Inc., Houston, Tex., U.S.A.) were added to a 1.5 ml tube and placed on ice. A 5×2×2 mm piece of frozen tissue sample was cut and ground in the extraction solution in the tube using a micro pestle. 500 μl of RNAzole™ and 80 μl of chloroform/isoamyl alcohol (24:1) were then added, vortexed for 10 secs and left on ice for 5 min. After centrifugation for 10 mins, 350 μl of the upper phase was transferred to a new tube containing 350 μl isopropanol and mixed by vortex. The tube was then placed on ice for 30 min and centrifuged at maximum speed for 20 min. The resulting pellet was washed twice with 70% ethanol, dried briefly and dissolved in 50 μl of DEPC-treated water and 25 u (1 μl) RNAguard® (a nuclease inhibitor, Pharmacia Biotech AB, Uppsala, Sweden).

For each set of RNA isolations made, a negative control (no tissue added) was processed in the same way.

Preparation of cDNA

The RNA sample obtained above was heat denaturated at 90° C. for 3 min and quenched on ice. 37.5 μl of 2×cDNA mix (90 mM Tris-HCl, pH 8.3, 138 mM KCl, 18 mM MgCl_{2+B, 30} mM DDT, 3.6 mM DATP, dCTP, dTTP, dITP and 0.9 mM dGTP, 0.152 U_{A260} pd(N)₆), 10 μl of MMULV reverse transcriptase (RT) (200 u) and 2.5 μl of RNAguard® (62.5 u) were mixed in a tube and 25 μl of the denaturated RNA sample were added. After incubating for 1 h at 37° C., the cDNA reaction was heat denaturated at 90° C. for 3 min, and the cDNA samples were stored at −20° C.

For each set of cDNA reactions made, a negative control (25 μl of water instead of RNA sample) was processed in the same way.

PCR Amplification of cDNA

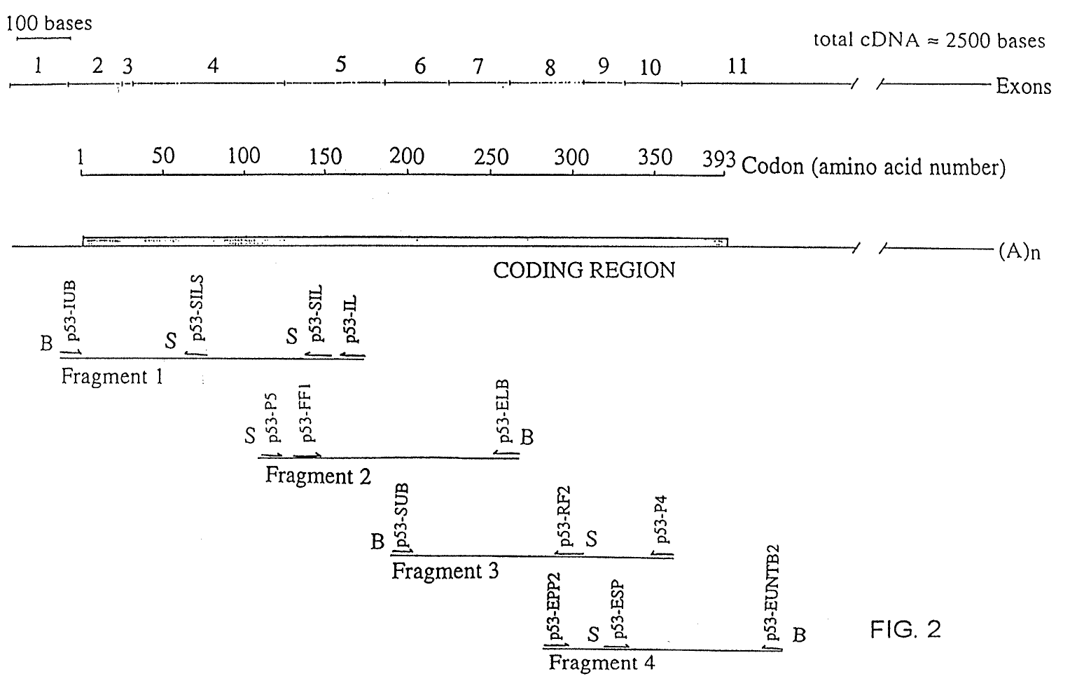

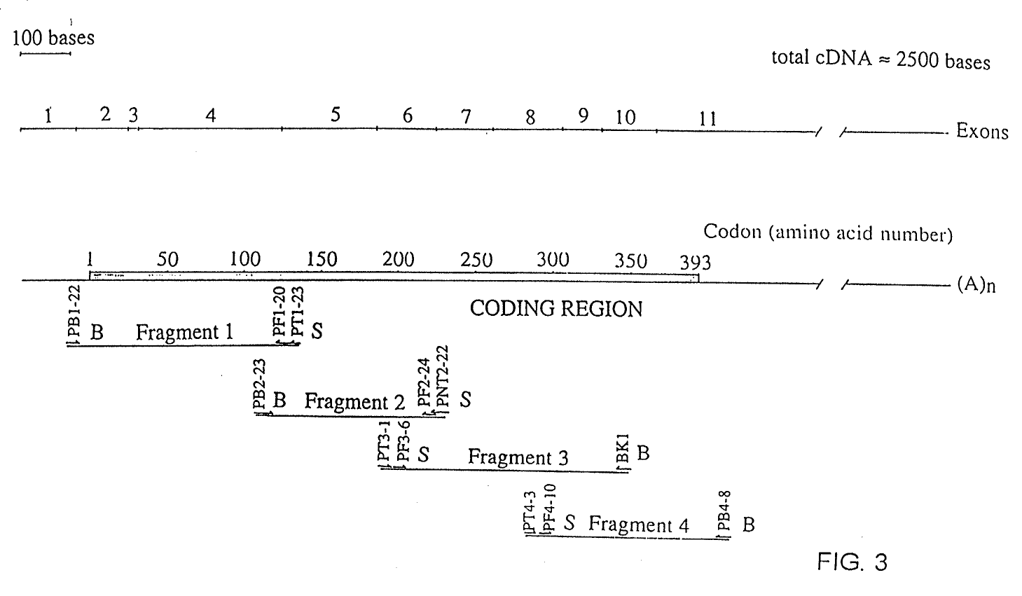

Four different fragments of the cDNA from each of the two sample groups (Fragments 1 to 4 in FIG. 2 and FIG. 3, respectively) were amplified in separate reactions, using the PCR primers shown in the sequence listing at the end of the description for the cDNA derived from the first group of 107 patients, and the PCR primers shown also at the end of the description for the cDNA derived from the second group of 292 patients. Each reaction was performed in a Perkin Elmer 9600 PCR machine (Perkin Elmer-Cetus, Emeryville, Calif., U.S.A.) as follows:

In a 0.2 ml tube were mixed 5 μl of PCR II buffer (10×) (Perkin Elmer-Cetus, Emeryville, Calif., U.S.A.), 5 μl of 5′-primer (1 pmol/μl), 5 μl of 3′-primer (1 pmol/μl), 1.2 μl of 25 mM MgCl₂, 28 μl of water and 0.8 μl of AmpliTaq polymerase (4 u) (Perkin Elmer-Cetus, Emeryville, Calif., U.S.A.). 5 μl of cDNA sample or 5 μl of negative control sample were added (total PCR reaction =50 μl). The samples were cycled 38× with the AUTO profile: 94° C. for 15 sec, 58° C. for 30 sec, 72° C. for 45 sec. The amplification reaction was ended with a 5 min HOLD at 72° C. and linked to HOLD file 4° C. [symbol] ∞. Purity, quality and quantity were checked by running 5 μl of the PCR reaction on a 1% agarose gel with 0.2 μg of the 100 Base-Pair Ladder (molecular weight marker, Pharmacia Biotech AB, Uppsala, Sweden) as reference.

DNA Sequencing

Sequencing of the four DNA fragments obtained above were performed in an A.L.F.™ DNA Sequencer (Pharmacia Biotech AB, Uppsala, Sweden) . The sequencing reactions were performed using comb-like polystyrene manifolds and corresponding well plates as described in our WO 94/11529. Each comb had 8 teeth, and the well plates were of two types, one type with wells designed to receive four comb teeth, below referred to as “four teeth well”, and a second type with each well designed to receive a single comb tooth, below referred to as “one tooth well”.

The following fragments of the p53 gene were sequenced, using the sequencing primers shown in FIGS. 2 and 3:

Primer set 1 for cDNA derived from the first group of patients

| Designation | Exons | Base pairs |
|---|---|---|
| SILS | 2 to half 4 | 316 to 136 |
| SIL | 2 to 5 | 575 to 136 |
| FF1 | 5 to 8 | 523 to 936 |
| RF2 | 6 to 9 | 1080 to 739 |
| ESP | 9 to 11 | 1060 to TGA (stop) |

Primer set 2 for cDNA derived from the second group of patients

| Designation | Exons | Base pairs |
|---|---|---|
| PF1-20 | 2 to 5 | 521 to 136 |
| PF2-24 | 5 to 7 | 793 to 458 |
| PF3-6 | half 6 to 10 | 741 to 1179 |
| PF4-10 | 9 to 11 | 1032 to TGA (stop) |

1. The PCR product obtained above (40 μl ) was transferred to a “four teeth well” containing 80 μl of BW buffer (1×TE, 2 M NaCl). Mixing was performed by pipetting, avoiding bubbles. The avidin-coated tips of a comb were inserted into the well and dipped a couple of times to improve the capture of biotinylated PCR product to the comb and were then left at room temperature for at least 60 min.

2. The comb was then moved to another “four teeth well” containing 100 μl of 0.1 M NaOH and incubated for 5 min for elution of the unbound DNA strands. The comb was then washed once in 100 μl of 0.1 NaOH, once in 100 μl of TE buffer and finally once in 100 μl of ultra-pure water.

3. To a new “four teeth well” were added 104 μof water, 12 μl of 10×Annealing buffer (AutoRead™ Sequencing Kit, Pharmacia Biotech AB, Uppsala, Sweden), 4 μl of a 1 pmol/μl fluorescein-labelled sequencing primer (see FIGS. 2 and 3), and the comb was inserted into the well. The annealing mix was heated to 55° C. for 5 min and then left at room temperature for at least 10 min.

4. From previously prepared master mixes of Sequence-mix (2 μl 10×Annealing buffer, 1 μl Extension buffer (AutoRead ™ Sequencing Kit, Pharmacia Biotech AB, Uppsala, Sweden), 4 μl d/ddNTP mix, 12 μl water, 1 μl (2 u) T7 polymerase diluted in Enzyme-dilution buffer (AutoRead™ Sequencing Kit, Pharmacia Biotech AB, Uppsala, Sweden)) for each d/ddNTP, stored on ice, with the T7 enzyme added as late as possible, 20 μl of each respective sequence-mix were dispensed in individual “one tooth wells”. Immediately after that, the comb with annealed primer was inserted into the wells, incubated for 5min at 37° C. and then placed on ice.

5. The loading wells of an A.L.F.™ DNA Sequencer gel prewarmed to 45° C. were rinsed and loaded with 15 μl Stop solution (AutoRead™ Sequencing Kit, Pharmacia Biotech AB, Uppsala, Sweden) to each well. The comb was removed from the “one tooth wells” above and inserted into the rinsed loading wells and left for 10 min to release the respectively terminated primer extension products. The comb was then carefully removed and the electrophoretic separation and detection process of the A.L.F.™ DNA Sequencer was started.

Results

317 of the patients whose tumour samples were analyzed as described above met the set test criteria for the evaluation of the results, and their test data were processed further with regard to other patient data, such as node status, adjuvant therapy, months of relapse-free survival, and death in breast cancer. The results obtained in this evaluation are presented below for, on one hand, (i) node negative patients with and without mutations, respectively, and (ii) node positive patients with and without mutations, respectively, and, on the other hand, the influence of p53 mutations in evolutionarily conserved regions versus mutations outside such regions. Also the exact position of p53 mutations and the corresponding amino acid change will be described for a number of (i) node negative and (ii) node positive breast cancer patients.

### Effect of Adjuvant Therapy

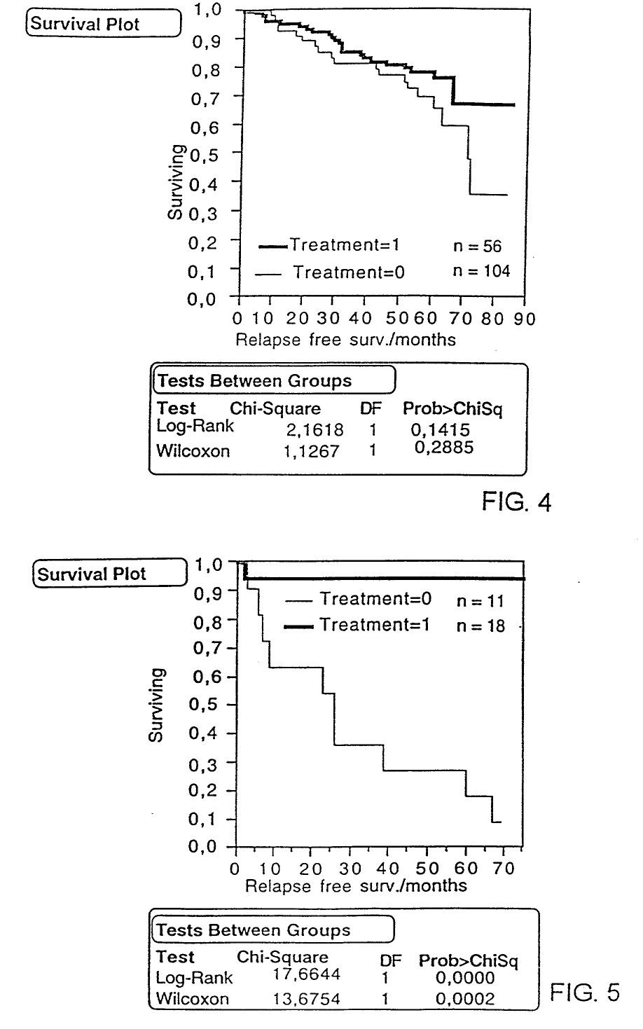

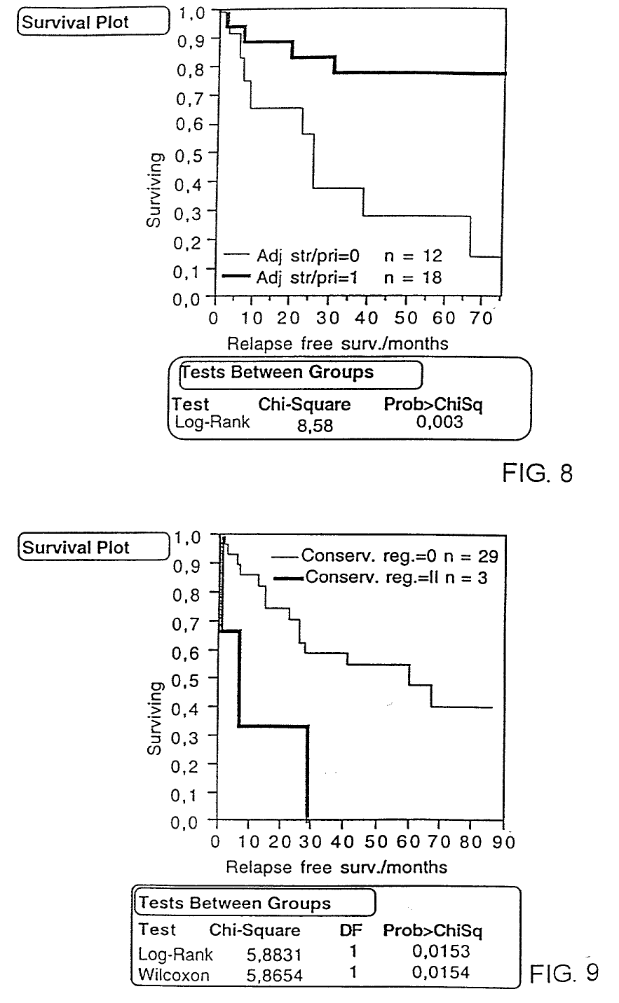

The effect of adjuvant therapy, i.e. radiation and/or polychemotherapy (premenopausal patients) or hormonal therapy (postmenopausal patients), after surgical removal of the tumour was studied. The results are summarized below and presented in FIGS. 4 to 6. A special study on the effect of loco-regional radiotherapy was also carried out on node-negative breast cancer patient. The results are presented in FIGS. 7 and 8.

### Node Negative Breast Cancer Patients

A. Without p53 Mutation

Patients given adjuvant therapy did not seem to perform better than patients not given adjuvant therapy, as is shown in FIG. 4.

B. With p53 Mutation

Patients not given adjuvant therapy had a very poor prognosis, whereas patients given such therapy had a long relapse-free survival, as is demonstrated in FIG. 5.

### Node Positive Breast Cancer Patients

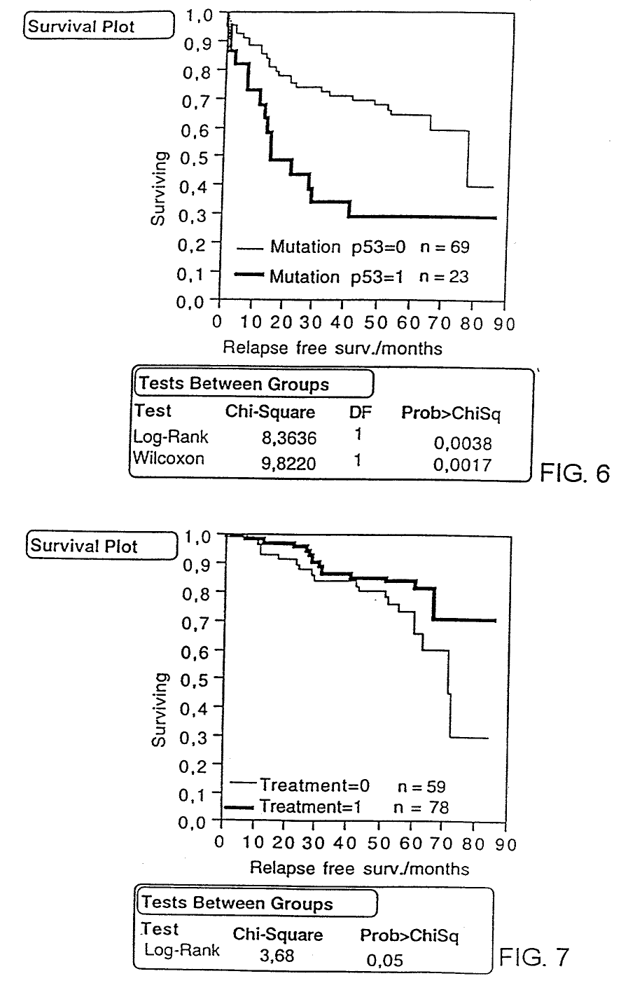

These patients, who all received adjuvant therapy, had a poor prognosis irrespective of whether they had a p53 mutation or not, better, however, for patients without a p53 mutation, as is shown in FIG. 6. A more efficient therapy is therefore required, especially for node positive patients with a p53 mutation.

### Node Negative Breast Cancer Patients

A. Without p53 Mutation

In patients with wild type p53 there was no significant difference in relapse-free survival between patients who had received postoperative loco-regional radiotherapy and those who had not, as shown in FIG. 7.

B. With p53 Mutation

The relapse-free survival was significantly better for node-negative patients with p53 mutations who had received postoperative loco-regional radiotherapy. See FIG. 8.

### P53 Mutation in a Conserved Region

The effect of a p53 mutation in an evolutionarily conserved region (for the locations of the conserved regions in the p53 gene it is referred FIG. 1) versus a mutation outside the conserved regions was studied. The results are summarized below and presented in FIGS. 9 to 12.

### Conserved Region II

As is demonstrated in FIG. 9, patients with a p53 mutation in conserved region II had a much poorer prognosis than patients with a mutation outside conserved regions.

### Conserved Region III

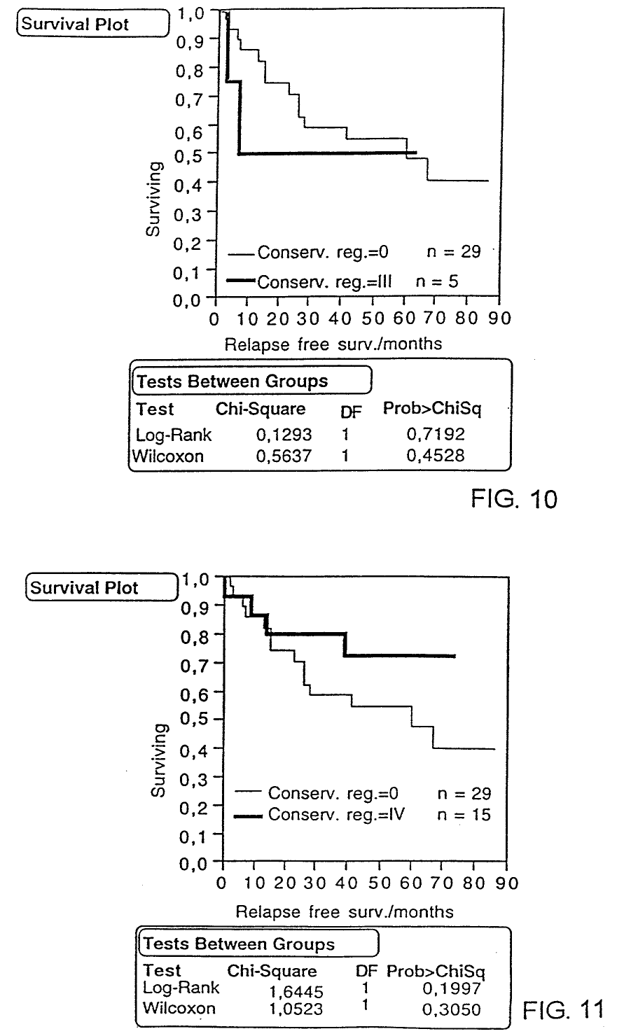

As is demonstrated in FIG. 10, there is no significant difference in relapse-free survival rate for patients with a mutaion in conserved region III compared to patients with a mutation outside conserved regions.

### Conserved Region IV

As is demonstrated in FIG. 11, a mutation in conserved region IV is not more serious to the patient than a mutation outside conserved regions.

### Conserved Region V

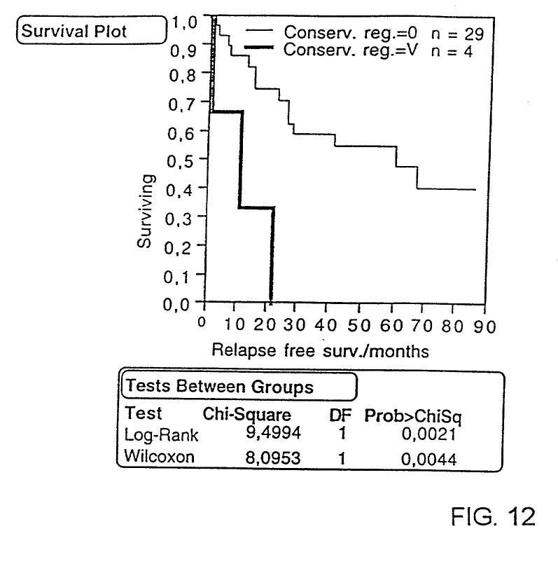

As is demonstrated in FIG. 12, patients with a p53 mutation in conserved region II had a much poorer prognosis than patients with a mutation outside conserved regions.

### Positions of Mutations in the P53 Gene

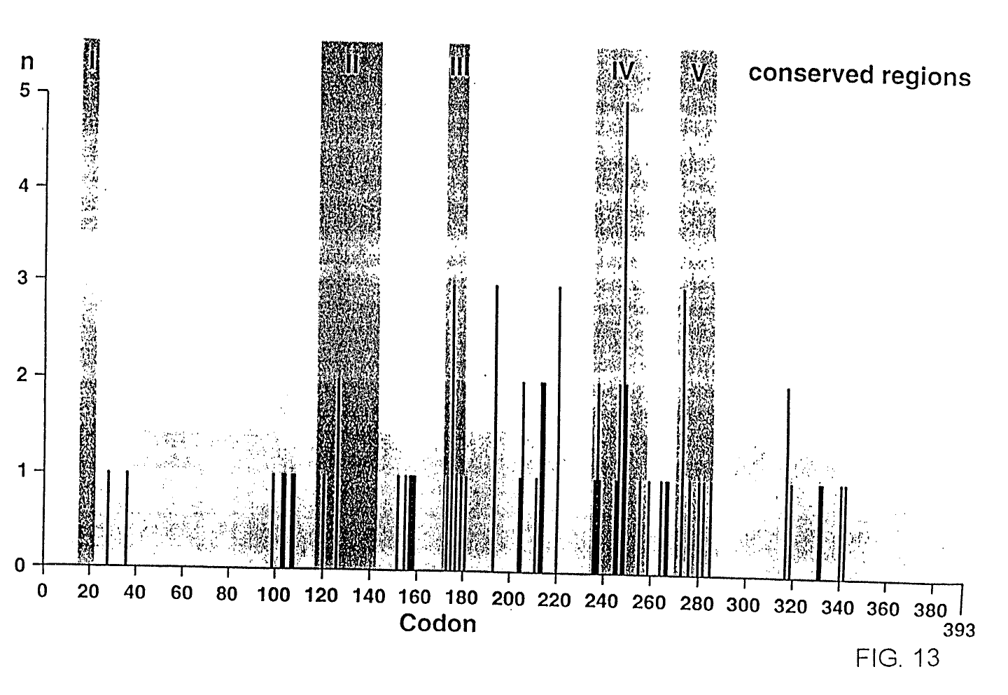

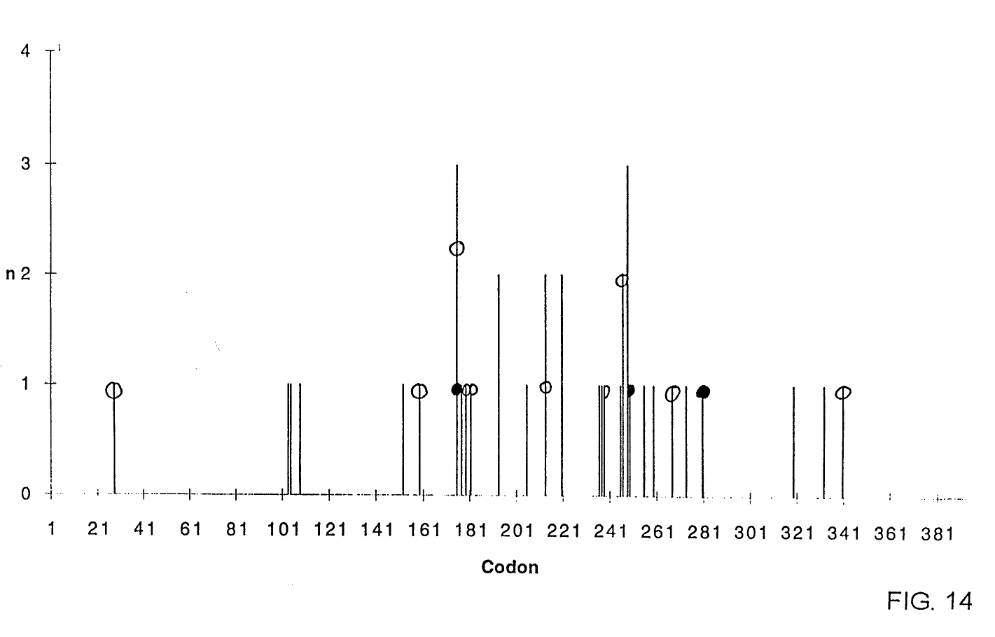

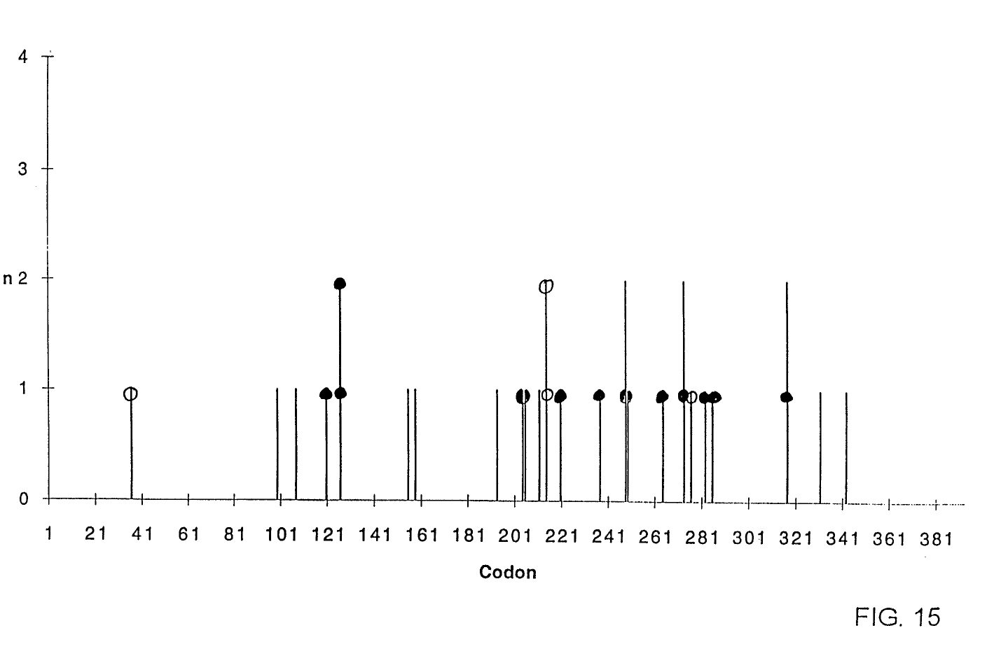

FIG. 13 shows the codon positions of mutations found in a number of samples from a group of patients, and FIG. 14 shows the codon positions of mutations found in a number of samples from node negative patients and FIG. 15 from node positive patients. An unfilled ring (o) indicates that the patient had a relapse, and a filled ring (●) that the patient died of breast cancer. A comparison of FIGS. 14 and 15 indicates that basically the positions of serious mutations for node negative patients differ from the positions of serious mutations for node positive patients.

The amino acid changes related to the mutations shown in FIGS. 14 and 15 are given in Tables 1 and 2 below.

TABLE 1
Node negative patients

| Amino acid | Transition |
|---|---|
| 28 | Glu→Ala |
| 103 | 19 bp deletion |
| 104 | Gln→stop |
| 108 | 11 bp deletion |
| 152 | Pro→Leu |
| 159 | Ala→Val |
| 175 | Arg→His (3×) |
| 177 | 9 bp deletion |
| 179 | His→Gln |
| 181 | Arg→His |
| 193 | His→Arg |
| 193 | His→Leu |
| 205 | Tyr→Cys |
| 213 | Arg→stop (2×) |
| 220 | Tyr→Cys (2×) |
| 236 | Tyr→Cys |
| 237 | Met→Ile |
| 238 | Cys→Phe |
| 245 | 3 bp insertion |
| 246 | Met→Thr |
| 248 | Arg→Gln (2×) |
| 248 | Arg→Trp |
| 249 | Arg→Ser |
| 255 | Ile→Phe |
| 259 | 1 bp deletion |
| 267 | 9 bp deletion |
| 273 | Arg→CyS |
| 280 | Arg→Gly |
| 319 | 1 bp deletion |
| 332 | 14 bp insertion→splice 9/10 |
| 340 | 2 bp insertion |

TABLE 2
Node positive patients

| Amino acid | Transition |
|---|---|
| 36 | Pro→Leu |
| 99 | 1 bp deletion |
| 107 | 2 bp deletion |
| 120 | 200 bp deletion |
| 126 | 21 bp deletion→splice 4/5 |
| 126 | 21 bp deletion/splice 4/5 |
| 155 | Thr→Ile |
| 158 | Arg→Pro |
| 193 | His→Arg |
| 204 | Glu→stop |
| 205 | Tyr→Cys |
| 211 | Thr→Ala |
| 214 | 2 bp deletion |
| 214 | His→Arg |
| 220 | Tyr→Cys |
| 237 | Met→Ile |
| 248 | Arg→Gln |
| 248 | Arg→Trp |
| 249 | Arg→Ser |
| 264 | 3 bp deletion |
| 273 | Arg→Cys |
| 273 | Arg→Leu |
| 276 | Ala-Gly |
| 282 | Arg→Pro |
| 285 | Glu→Lys |
| 317 | 1 bp insertion |
| 317 | Gln→stop |
| 331 | Gln→stop |
| 342 | Arg→stop |

As has been demonstrated above, very valuable information on the tumour status may be obtained by sequencing at least large parts of the p53 gene in a neoplastic sample.

### EXAMPLE 2

Comparison between Sequence Based Diagnosis (SBD) and immunohistochemical (IHC) Staining Method

Substantially all the patient samples tested above were also subjected to an immunohistochemical (IHC) testing procedure as follows.

Freshly resected breast tumour tissue was fixed in formalin for 1 h, dehydrated in 60% ethanol for 30 min, dehydrated in 80% ethanol for 1 h, dehydrated in 95% ethanol for 30 min, dehydrated in 99% ethanol for 3.5 h, dehydrated in xylene for 2.5 h, and treated with paraffin for 3 h. All steps were performed in Tissue-Vek VIP over-night. Finally, the tissue sample was embedded in paraffin blocks possible to store for longer periods of time and from which it was possible to cut 3-5 μm sections.

The sections were then de-paraffinized in xylene and rehydrated in 99% ethanol, 95% ethanol, 80% ethanol, and finally distilled water.

Prior to staining for p53 protein, the sections were pretreated in a microwave oven to make the p53 antigen accessible for the antibody using the following protocol:

Three jars were placed in a water bowel, each containing 50 ml of 10 mM citrate buffer, pH 6.0. The samples were irradiated 3×5 min at 700 W, adjusting the liquid layer between irradiations. Finally, the jars were cooled in distilled water.

The staining procedure was performed in a Ventana ES Automated Immunohistochemistry Instrument (Annex, Helsinki, Finland). Briefly, microscopic slides were treated with mouse monoclonal antibodies directed against the wildtype and mutant forms of p53 (cl 1801) at dilutions of +E, fra 1/100(Bio-Zac AB, Jarfalla, Sweden). After rinsing in APK buffer, the bound antibodies were visualized using the Ventana DAB detection kit consisting of the sequential application of biotin-labelled secondary antibodies against mouse immunoglobulins, avidin-labelled horse radish peroxidase, H₂0₂, and finally diamino benzidine (DAB) generating a precipitating enzyme product. Between each step appropriate rinsing of the samples was performed.

The sections were then rinsed in warm tap water for 15 min. Finally, the sections were dehydrated in 99% ethanol, 95% ethanol, and 80% ethanol, respectively, and distilled water and finally cleared in xylene and mounted in Pertex (Histolab).

Comparison Between IHC and SBD

For the whole sample material, the following result was obtained by IHC and SBD, respectively.

SBD

| | | + | − | |
|---|---|---|---|---|
| IHC | + | 40 | 18 | 58 |
| | − | 18 | 209 | 227 |
| | | 58 | 227 | 285 |

The 40 patient samples testing positive in both IHC and SBD comprise 3 samples where more extensive genetic changes have occurred, viz.

| Codon | Change |
|---|---|
| 267 | 9 bp deletion |
| 245 | 3 bp insertion |
| 126 | 21 bp deletion |

The above three changes are all in-frame mutations.

The 18 patient samples which are negative in IHC and positive in SBD comprise 11 samples which exhibit considerable changes, viz.

| Codon | Change |
|---|---|
| 213 | Arg→stop |
| 204 | Glu→stop |
| 341 | Arg→stop |
| 264 | 3 bp deletion |
| 120 | ≈200 bp deletion |
| 317 | Glu→stop |
| 165 | Glu→stop |
| 108 | 11 bp deletion |
| 126 | 21 bp deletion |
| 103 | 19 bp deletion |
| 177 | 9 bp deletion. |

When the samples are divided into node positive and node negative patients, the following results are obtained:

Node negative
SBD

| | | + | − | |
|---|---|---|---|---|
| IHC | + | 22 | 10 | 32 |
| | − | 8 | 142 | 150 |
| | | 30 | 152 | 182 |

Of the 22 patient samples which are positive in both IHC and SBD, 2 samples exhibit great changes, whereas 4 of the 8 samples that are negative in IHC but positive in SBD exhibit great changes.

Node positive
SBD

| | | + | − | |
|---|---|---|---|---|
| IHC | + | 15 | 4 | 19 |
| | − | 10 | 60 | 70 |
| | | 25 | 64 | 89 |

Of the 15 patient samples which are positive in both IHC and SBD, 1 sample exhibits a great change, whereas 7 of the 10 samples that are negative in IHC but positive in SBD exhibit great changes.

The above results clearly indicate that IHC will fail to identify a considerable number (approximately one third of the tested samples) of patients with p53 mutations, especially when great genetic changes are involved. This may be particularly disadvantageous for the previously mentioned subgroup of node negative breast cancer patients with a p53 mutation, which patients have a very good prognosis when receiving appropriate adjuvant therapy after surgery.

Sequence Listing

Representation of the DNA sequences of the primers schematically indicated in FIG. 2

Position in p53
in relaiton to A
in start codon ATG

Fragment 1
PCR primers

| | | |
|---|---|---|
| p53-IUB: | B-5′-GTG CTT TCC ACG ACG GTG A-3′ | −65/−47 |
| p53-IL: | 5′-TCA TGT GCT GTG ACT GCT TGT AG-3′ | 507/485 |

Sequence primer

| | | |
|---|---|---|
| p53-SIL: | F-5′-GGC GGG GGT GTG GAA TCA A-3′ | 457/439 |
| p53-SILS: | F-5′-TCT GGC ATT CTG GGA GCT TCA TC-3′ | 202/180 |

Fragment 2
PCR primers

| | | |
|---|---|---|
| p53-ELB: | B-5′-CCG TCC CAG TAG ATT ACC AC-3′ | 800/781 |
| p53-P5: | 5′-GTT TTC CGT CTG GGC TTC TT-3′ | 321/340 |

Sequence primer

| | | |
|---|---|---|
| p53-FF1: | F-5′-CTG TGA CTT GCA CGT ACT CCC CTG CCC-3′ | 361/387 |

Fragment 3
PCR primers

| | | |
|---|---|---|
| p53-P4: | 5′-TAG ACT GAC CCT TTT TGG ACT TC-3′ | 1128/1106 |
| p53-SUB: | B-5′-CGT GTG GAG TAT TTG GAT GAC-3′ | 603/623 |

Sequence primer

| | | |
|---|---|---|
| p53-RF2: | F-5′-TGG TTT CTT CTT TGG CTG GGG A-3′ | 965/944 |

Fragment 4
PCR primers

| | | |
|---|---|---|
| p53-EPP2: | 5′-GCT TTG AGG TGC GTG TTT GTG-3′ | 805/825 |
| p53-EUNTB2: | B-5′-CTG TCA GTG GGG AAC AAG AAG-3′ | 1211/1191 |

Sequence primer

| | | |
|---|---|---|
| p53-ESP: | F-5′-GGA GCA CTA AGC GAG CAC TG-3′ | 904/923 |

B = Biotin
F = Fluorescein

Representation of the DNA sequences of the primers schematically indicated in FIG. 3

Position in p53
in relation to A
in start codon ATG

Fragment 1
PCR primers

| | | |
|---|---|---|
| PB1-22: | B-5′-GAC ACG CTT CCC TGG ATT GGC-3′ | −88/−28 |
| PT1-23: | 5′-GCA AAA.CAT CTT GTT GAG GGC A-3′ | 404/383 |

Sequence primer

| | | |
|---|---|---|
| PF1-20: | F-5′-CAG GGG AGTACG TGC AAG TCA CAG-3′ | 497/385 |

Fragment 2
PCR primers

| | | |
|---|---|---|
| PB2-23: | B-5′-GTT TCC GTC TGG GCT TCT TGC A-3′ | 322/439 |
| PNT2-22: | 5′-GGT ACA GTC AGA GCC AAC CTC-3′ | 689/669 |

Sequence primer

| | | |
|---|---|---|
| PF2-24: | F-5′-GCC AAC CTC AGG CGG CTC ATA-3′ | 677/657 |

Fragment 3
PCR primers

| | | |
|---|---|---|
| PT3-1: | 5′-TGG CCC CTC CTC AGC ATC TTA-3′ | 562/582 |
| BK1: | B-5′-CAA GGC CTC ATT CAG CTC TC-3′ | 1043/1024 |

Sequence primer

| | | |
|---|---|---|
| PF3-6: | F-5′-CGA GTG GAA GGA AAT TTG CGT-3′ | 585/605 |

Fragment 4
PCR primers

| | | |
|---|---|---|
| PT4-3: | 5′-CGG CGC ACA GAG GAA GAG AAT C-3′ | 843/864 |
| PB4-8: | B-5′-CGC ACA CCT ATT GCA AGC AAG GG-3′ | 1287/1264 |

Sequence primer

| | | |
|---|---|---|
| PF4-10 | F-5′-GGG GAG CCT CAC CAC GAG CTG-3′ | 876/896 |

B = Biotin
F = Fluorescein

1. A method for sequence-based diagnosis of a human neoplastic tissue, blood or other body fluid sample,by analysing from genomic DNA or cDNA derived from said neoplasia the DNA sequence of a gene encoding the cancer-related p53 protein for the presence of mutations therein, characterized by a) determining from the presence, nature and location of any such mutation or mutations the influence thereof on the biological function of the corresponding protein and thereby on the properties of the neoplasia, b) classifying the neoplasia into different subgroups depending on (i) the presence or not of a mutation, and (ii) whether the patient is node positive or not, and on the basis thereof prognosticating the development of the neoplasia and provide a guidance for adequate treatment of the patient.

2. The method of claim 1, characterized in that said properties of the neoplasia includes biological aggressiveness and/or metastatic potential.

3. The method of claims 1 or 2, characterized by analyzing a part or parts of the gene which encode at least one biologically functional domain of the cancer-related protein.

4. The method of claim 3, characterized in that said biologically functional domain includes a DNA binding domain and/or transactivation site.

5. The method of claim 3 or 4, characterized in that evolutionary conserved regions of the gene are analyzed.

6. The method of claim 1, characterized in that the neoplasia is a breast, lung, prostate, gastric, colorectal, melanoma or leukemia neoplasia.

7. The method of claim 6, characterized in that said sample originates from a breast neoplasia.

8. The method of claim 7, characterized in that the detection of the presence of a p53 mutation in a node negative patient tumour sample is indicative of the need of adjuvant therapy following surgical removal of the tumour.

9. The method of claim 8, characterized in that the adjuvant therapy is radiation or chemotherapy/hormone therapy.

10. The method of any one of claims 1 to 9, characterized in that it comprises one or more of the following steps: preparation of genomic DNA or cDNA, amplification of at least part of the cancer-related gene, processing of the cancer-related gene including sequencing reactions, and detection of the products from the sequencing reactions in an automated nucleic acid sequencer, computer software optionally being used to (i) track samples and control process steps and/or (ii) to aid in and/or interpret sequence data obtained.

11. A method of detecting mutations in a gene, characterized by comprising the steps of preparing genomic DNA or cDNA, amplifying at least part of the gene, processing the amplified DNA to produce sequencing reaction products, preferably by solid phase based techniques, detecting the sequencing reaction products in an automated nucleic acid sequencer to determine a DNA sequence or sequences of the p53 gene, and comparing the sequence or sequences with the corresponding wild type p53 gene sequence or sequences, computer software being used to (i) track samples and control process steps and/or (ii) to at least aid in interpreting sequence data obtained.

12. The method according to claim 11, characterized in that mutations are detected in a gene encoding the p53 protein.
